(19) United States
(12) Patent Application Publication
(10) Pub. No.: US 2009/0263798 A1
(43) Pub. Date: Oct. 22, 2009

(54) Method For Identification Of Novel Physical Linkage Of Genomic Sequences

(75) Inventors: Johannes Dapprich, Lawrenceville, NJ (US); Abram Gabriel, Princeton, NJ (US); Maitreya Dunham, Princeton, NJ (US)

Correspondence Address: CONNOLLY BOVE LODGE & HUTZ, LLP, P O BOX 2207, WILMINGTON, DE, 19899, US

(73) Assignees: Generation Biotech, LLC, Lawrenceville, NJ (US); Rutgers, the State University, New Brunswick, NJ (US); Princeton University, Princeton, NJ (US)

(21) Appl. No.: 12/300,657

(22) PCT Filed: May 14, 2007
(86) PCT No.: PCT/US07/11544

§ 371 Date: Nov. 13, 2008

Related U.S. Application Data
(60) Provisional application No. 60/800,426, filed on May 15, 2006.
(60) Provisional application No. 60/833,042, filed on Jul. 25, 2006.

(51) Int. Cl. C12Q 1/68

(52) U.S. Cl. 435/6

(57) ABSTRACT

The invention is directed to methods to identify the location in a genome of a nonfixed or multicopy genomic element using microarrays or sequencing.

### CROSS-REFERENCE TO RELATED APPLICATIONS

This application claims the benefit of U.S. Provisional Application No. 60/800,426, filed May 15, 2006, and U.S. Provisional Application No. 60/833,042 filed Jul. 25, 2006, both of which are herein incorporated by reference in their entireties.

### STATEMENT REGARDING FEDERALLY SPONSORED RESEARCH

The U.S. government may have certain rights in this invention as provided for by the terms of grants R44 AI 51036-02 and P50 GM071508, both awarded by the National Institutes of Health.

### FIELD OF THE INVENTION

The present invention relates to methods for identifying the presence and location of nucleic acid segments within a genome.

### BACKGROUND OF THE INVENTION

Whereas the location of most genomic sequences is fixed along a chromosome, some genomic elements are nonfixed or may occur in multiple copies. Nonfixed genomic elements, such as transposable elements, chromosomal rearrangement breakpoints, natural viral insertions, artificial insertion events such as insertional libraries, as well as other natural or induced recombination events, all can have unpredictable and unique sites of joining to chromosomal DNA. As such, these new linkages can have profound effects on genomes through altered gene expression and/or disease causation. Further, where such new linkages do not affect the phenotypic characteristics of the host, differences within a population (for example, plant strains) are only distinguishable at the molecular level.

However molecular analysis to determine the positions of nonfixed or copy number variable elements throughout the genome can be difficult or impossible to determine by sequence analysis due to the problem of properly assembling relatively short reads generated by random shotgun sequencing into their proper genomic context of potentially much larger repetitive elements, segmental duplications, translocations, inversions or other chromosomal rearrangements. This has become an acute problem for so-called “next-generation sequencing (NGS)” approaches that rely on the genome wide assembly of very short read lengths (typically 10-30 base pairs, sometimes 30-100 base pairs), especially in combination with more complex genomes, such as the human genome.

With respect to transposons, the genomes of all organisms studied have evidence of multiple invasions over evolutionary time by different classes of transposons. These multicopy genetic elements, first postulated by Barbara McClintock, are regulated at many levels to suppress their invasive potential, but their movement has been shown to result in genetic diseases in humans (Kazazian, 1998), hybrid dysgenesis and sterility in Drosophila (Engels, 1996), the spread of antibiotic resistance in bacteria (Kim et al., 1998) and insertional activation or inactivation of nearby genes. Their effects on host genomes can be more widespread and subtle. The presence of the L1 retrotransposon in the intron of a gene can affect its expression by slowing of transcription through the L1 sequence (Han et al., 2004). Polymorphic transposon sequences within genes can result in allele-specific alternative splicing patterns with formation of new exons (Sorek et al., 2002). Their multicopy nature and dispersion throughout genomes results in their appearance at breakpoints of gross chromosomal rearrangements, such as translocations, inversions, and deletions (Dunham et al., 2002; Lemoine et al., 2005; Yu and Gabriel, 2003; Yu and Gabriel, 2004).

These transposon associated rearrangements may be selectively advantageous, as has been shown by experimental evolution studies for yeast maintained in chemostat cultures with limiting nutrients (Dunham et al., 2002; Perez-Ortin et al., 2002). Thus the differences in placement of transposons in individual genomes could cause or at least correlate with phenotypic differences.

While whole genome sequencing can identify all transposable or multicopy elements in the specific genome under examination, the results may not apply to other strains of the same species. Since transposable elements may have profound impacts on their host genomes, the global position of all transposons in a specific genome, and the similarities or differences between individual genomes in a given species, can serve as a basis for understanding individual differences and adaptive potential. Thus methods for the simultaneous detection of the presence and location of transposons over an entire genome are needed. Furthermore, even if the presence of transposons does not correlate with phenotypic differences, whole genome methods for identifying strain-specific transposon polymorphisms would be useful in the yeast brewing and baking industries, in the grape industry, in the use of other plant species as a means for distinguishing different strains, or in the tracing of lineages in humans or any other species.

With regard to chromosomal rearrangements, it is well known that pharmaceutical drugs, chemicals and other environmental agents such as tobacco, radiation, sunlight, heavy metals, and stress, can cause chromosomal rearrangements, either by breaking and rejoining of DNA segments, or by inducing the movement of transposable agents. Tumor specific chromosomal rearrangements can have diagnostic and prognostic value. Methods for monitoring, quantifying and specifically characterizing the propensity of different agents to cause these gross chromosomal rearrangements over an entire genome are needed. In a similar vein, methods are needed for detecting specific rearrangement partners. Identification of potential subtle rearrangements in a specific tumor could be used to stage and identify tumors and predict their response to therapy and outcome.

With regard to natural viral insertions, it is known that many viral diseases involve integration of viral nucleic acid into the host genome. These include diseases caused by retroviruses such as HIV, HTLV-1 in humans, as well as BLV in cows, FLV in cats, Visna in sheep, and equine infectious anemia virus in horses. Such diseases also involve DNA viruses such hepatitis B, as well as certain viruses that can maintain latency by genomic integration, such as adenovirus, human papilloma virus, and measles virus. Certain plant viruses are also known to insert into genomes in random manners. Thus methods for genome wide detection of the presence and position of integration of natural viral insertions are needed as well as methods that reduce the complexity of a genomic sample.

Finally, genomes can be made to rapidly evolve under selective pressures. The resulting changes in the genome structure and organization can reveal novel metabolic and genetic pathways. Chromosomal changes that result in so-called ‘position-effect mutations’ can lead to changes in gene expression levels as well as their temporal or spatial activation (Scherer et al, 2004; Spitz et al., 2005) and may cause inherited or de-novo human diseases (Shaw et al., 2004; Stankiewicz et al., 2002). Phenotypic information about gene function often is sought through the analysis of loss- or gain-of-function mutations resulting from DNA insertions. Many methods for generating populations comprising individuals with one or more mutations involve introduction and random insertion of unstable genomic elements (for example, transposons). Transposons, reporter cassettes, gene traps, promoter traps, and Agrobacterium T-DNAs all have been used as insertional mutagens in different organisms. However, the identification of insertion sites remains a methodological challenge in insertional mutagenesis. Thus methods for genome wide detection of the presence and position of an insertion event are needed.

The challenge for identifying the location of nonfixed, multicopy or randomly inserted genomic elements is the identification of the sequences which flank these genomic elements. This is true even though the nucleic acid sequence of the genomic element is known. DNA sequences flanking insertions have been identified by plasmid rescue or amplified by several semispecific PCR methods, such as inverse PCR, adapter-ligation PCR, vectorette PCR, or thermal asymmetric interlaced-PCR (TAIL-PCR). Although laborious and expensive, sequencing of cloned or PCR-amplified flanking fragments unequivocally identifies insertion sites, and databases of insertion-site sequences have been established for some genomes. However, all of these methods suffer from either a limitation that they permit screening for insertions in only one or a small number of genes at a time, or require use of semispecific PCR, which can be expensive, time-consuming, biased and incomplete.

Likewise, the proper assembly of genomic sequences that contain copy number variants or other rearrangements can be very difficult. The detection of gross chromosomal rearrangements in the genome of patients with genetic diseases by oligonucleotide microarrays or fluorescence in situ hybridization (FISH) is cumbersome and typically limited to a region of about 10-20 kilobases near a breakpoint. The routine assembly of larger blocks of contiguous, intergenic haplotype information from individual samples has been unattainable using current systems, and no solutions exist to deconvolute complex genomic regions related to copy number variations, repetitive elements and segmental duplications in a high-throughput mode. Therefore a need exists for methods that combine the flexibility of current genome analysis methods with the more informative content typically achieved only by manual, laborious screening methods.

### SUMMARY OF THE INVENTION

The invention is based on the discovery of a method for rapidly and economically identifying the location in a genome of a nonfixed or multicopy genomic element of interest. The method involves isolating a genomic nucleic acid fragment that contains the genomic element and a flanking sequence from the genome, labeling the isolated fragment to form a labeled probe, and applying the labeled probe to a sufficiently dense genomic microarray such that specific binding of the probe to one or more positions on the microarray can be determined and thus the location of the genomic element of interest can be determined. Alternatively, the labeling of the isolated fragments may occur after immobilization as part of a sequencing process, such as by successively attaching individual nucleotides to template fragments on a surface and thereby determining their sequence.

Other features and advantages of the invention will be apparent from the following detailed description and claims.

### BRIEF DESCRIPTION OF THE DRAWINGS

FIG. 1: A general schematic diagram of the steps involved in extracting, labeling and identifying the position of repetitive regions from a genome. The thick rectangle in step 1 is the repetitive element. The triangular and circular lollipops in step 6 represent differentially labeled nucleotides.

FIG. 2: A graph showing the log₂ ratio of hybridization for each feature along each chromosome plotted in genome order using the TreeView Karyoscope function. FIG. 2 illustrates the identification of a unique Ty1 element in otherwise isogenic strains. Two isogenic yeast strains (Fy5 and FY2) differ only by the presence of a Ty1 insertion in chromosome V within the URA3 gene in FY2. After labeling transposon extracted DNA from FY2 with Cy3 (below the horizontal lines) and transposon extracted DNA from FY5 with Cy5 (above the horizontal lines) the labeled DNA is hybridized to an Agilent Whole Genome Array with >40,000 unique features. The one region of significant differential hybridization is marked with an arrow.

FIG. 3: A graph showing the log₂ ratio of hybridization for each feature along each chromosome plotted in genome order using the TreeView Karyoscope function showing validation of whole genome transposon analysis using two sequenced strains of S. cerevisiae (A) Whole genome comparison of full-length Ty1 and Ty2 elements from yeast strains RM11 and S288c after hybridization to the same Agilent Whole Genome array. Black circles refer to the position of Ty1 or Ty2 full-length elements annotated for S288c in SGD. Triangles refer to full-length Ty2 elements identified in the sequence of RM11. Peaks above the horizontal lines correspond to potential Ty1 or Ty2 elements present in S288c while peaks below the horizontal lines correspond to potential Ty1 or Ty2 peaks present in RM11. (B) Comparison of location of Ty1 full-length elements (peaks below the horizontal lines) and Ty2 full-length elements present (peaks above the horizontal lines) in S288c.

FIG. 4: Comparison of full-length Ty1 and Ty2 elements on chromosome XV in strains S288c, CEN.PK, and W303. Rows 1, 2, 3, 5, 6, and 7 are based on transposon extraction data from Agilent Whole Genome arrays. Rows 4 and 8 correspond to Affymetrix tiling arrays probed with either CEN.PK DNA or W303 genomic DNA. For rows 1, 2, 5, and 6, digested genomic DNA as noted was extracted with either the set of Ty1-specific or Ty2-specific probes. For rows 3 and 7, digested genomic DNA was extracted with the set of common Ty1 and Ty2 probes. Grey horizontal lines above and below the central line for each chromosome correspond to a 3-fold ratio of signal intensity. In rows 4 and 8, light rectangles correspond to regions of CEN.PK and W303, respectively, derived from its S288c parent. In row 4, dark rectangles correspond to regions of CEN.PK derived from its non-S288c parent. In row 8, dark rectangles correspond to regions of W303 derived from its non-S288c parent. Row 1 is S288c, Ty1-peaks below the line and Ty2 peaks below the line; Row 2 is CenPK, Ty1-peaks below the line and Ty2 peaks below the line; Row 3 is CenPK Ty1 and Ty2 peaks below the line and S288c Ty1 and Ty2 peaks above the line; Row 4 is CenPK based on Affymetrix tiling array. Row 5 is S288c, Ty1-peaks below the line and Ty2 peaks below the line; Row 6 is W303, Ty1-peaks below the line and Ty2 peaks below the line; Row 7 is W303 Ty1 and Ty2 peaks below the line and S288c Ty1 and Ty2 peaks above the line; Row 8 is W303 based on Affymetrix tiling array.

FIG. 5: Based on microarray analysis of the uncharacterized SKI genome, the position of Ty1 or Ty2 elements and Ty3 LTR elements are shown. Ty1s are shown as circular lollipops above the horizontal lines; Ty2 are shown as triangular lollipops above the horizontal lines; Ty3 LTRs are shown as hexagonal lollipops below the horizontal lines.

FIG. 6: (A) Positions of 5 independent pooled artificial transposons from a yeast insertion library were determined after extracting StuI digested yeast genomic DNA with probes designed to correspond to either strand at the 5′ or 3′ end of URA3, labeling with Cy3 or Cy5, respectively, and hybridizing to an Agilent Whole Genome array. Arrows signify locations of significant differential hybridization. “URA3” refers to the actual URA3 locus on chromosome V. Vertical lines above and below the horizontal for each chromosome represent the log₂ ratio of hybridization intensity for Cy5 vs. Cy3 at each feature along the Agilent Yeast Whole Genome array. (B) An enlargement of the region detected on chromosome XI, showing the structure of the artificial transposon, its unique StuI site, the bases covered by the oligonucleotides in the features on either side of the transition from significant differential Cy5 labeling to Cy3 labeling, and the position of the actual insertion. Grey horizontal lines above and below the central line for each chromosome correspond to a 3-fold ratio of signal intensity.

FIG. 7: Region-specific extraction (RSE) of a segmental duplication with surrounding sequence context. Four RSE probes were used in separate experiments to isolate only one of two homologous regions on chromosome 6 (FIG. 7a). The probes target single nucleotide polymorphic markers (“SNPs”) that are unique to the respective copy and thereby distinguish the two copies which are separated by an ˜68 kb intervening sequence. The length of the input DNA was 50 kb. The typing results show the selective isolation of only one of the duplicate copies depending on where the probes target the region (FIG. 7b).

### DETAILED DESCRIPTION OF THE INVENTION

In one aspect the method of the invention comprises three steps.

The first step involves selective isolation of genomic nucleic acid fragments comprising at least a portion of the known sequence of a genomic element of interest and a flanking sequence (i.e., a flanking element) from a population of genomic nucleic acid fragments. In particular, a sample of genomic nucleic acid fragments (previously prepared from a population of genomic nucleic acid molecules) is contacted with a targeting element. Because the targeting element is capable of selectively binding to a known nucleotide sequence in the genomic element, when the genomic nucleic acid fragments are contacted with the targeting element, a complex is formed between the targeting element and a genomic nucleic acid fragment comprising the desired genomic element.

The targeting element either has a separation group already attached before it is contacted with the genomic nucleic acid fragments or, if it does not, a separation group is attached after contacting the sample of genomic nucleic acid fragments with the targeting element. The targeting element-genomic nucleic acid fragment complex is immobilized via binding or association of the separation group to a substrate. It is thereby separated from or purified away from the other non-complexed genomic nucleic acid fragments.

The second step of the inventive method involves preparation of labeled polynucleotide probes (capable of hybridizing to the microarray used in the third step) based on the captured polynucleotide sequence (i.e., the genomic nucleic acid fragment(s) of interest isolated in the first step). In particular, a method of linear amplification is used to prepare labeled probes using the isolated genomic nucleic acid fragment(s) from the first step as template. A targeting element, if a polynucleotide, and if extended in the first step to contain a flanking element strand complementary to the flanking element strand present in the complexed genomic nucleic acid fragment, may also serve as a template for labeled polynucleotide probes. The preparation of labeled probes can optionally include multiple, distinguishable labels for different bases of the template that permit the determination of the sequence of the labeled probes. A number of different labeling strategies generally employed for nucleic acid sequencing purposes are known to the skilled artisan.

In the third step, the labeled probes from the second step are applied to an array comprising discrete immobilized oligonucleotides having sequences corresponding to known genomic sequences. In an alternative embodiment, labeled probes are applied to an array comprising spotted polynucleotides of known sequence (cDNAs, PCR products, BACs, YACs, etc.). Detection of a signal from a bound labeled probe indicates that the nucleotide sequence of the immobilized oligonucleotide (or polynucleotide) corresponds to a sequence which flanks the genomic element of interest. Because the oligonucleotides (polynucleotides) immobilized to the array uniquely identify specific locations within the genome, a positive signal also indicates that a genomic element of interest is present at that location in the genome.

In a different embodiment of the invention, the second step involves the immobilization of the captured genomic nucleic acid fragment(s) of interest isolated in the first step by means of hybridization to a surface such as a microarray, microparticles or various semi-solid support materials such as gel matrices. A method of linear amplification is then used in a third step to prepare labeled probes or primer extension products using the isolated genomic nucleic acid fragment from the first step as template. Similar embodiments are frequently used in conventional and so-called ‘next generation’ sequencing approaches. See, for example, WO2006084132, WO9001562, US Pat. App. 20050244863, Cohen, J., MIT Technology Review magazine: issue May/June 2007.

Alternatively, these steps can also be interchangeably combined with each other in order to provide 1) a sequence-specific immobilization of the targeted and flanking sequence to pre-defined array positions, followed by 2) extension and sequencing of the immobilized template. In this way, the overall genomic context of the captured sequence can be encoded through the capture position on the array (as described in the transposon examples) and the high resolution information of the flanking sequence can be identified by a subsequent labeling and sequencing step. A related approach is described in US Patent Application 20050244863.

In various embodiments, the method disclosed herein may be used for manual operation, such as involving use of a prepackaged kit of reagents, and also for automated high-throughput operation. The inventive methods described here differ from previous approaches in not requiring ligation or PCR amplification, making the present methods simpler, more robust, and freer from amplification bias.

### Definitions and General Terms

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present Specification, including definitions, will control. In addition, the materials, methods, and examples are illustrative only and not intended to be limiting.

As used in the specification and claims, the singular form “a,” “an,” and “the” include plural references unless the context clearly dictates otherwise. For example, the term “a genomic DNA fragment” includes a plurality of genomic DNA fragments.

The practice of the present invention may employ, unless otherwise indicated, conventional techniques of organic chemistry, polymer technology, molecular biology (including recombinant techniques), cell biology, biochemistry, and immunology, which are within the skill of the art. Such conventional techniques include polymer array synthesis, hybridization, amplification, and detection of hybridization using a label. Specific illustrations of suitable techniques can be had by reference to the examples hereinbelow. However, other equivalent conventional procedures can, of course, also be used. Such conventional techniques can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press).

Methods and techniques applicable to array synthesis have been described in U.S. Pat. Nos. 5,143,854, 5,242,974, 5,252,743, 5,324,633, 5,384,261, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, and 6,090,555.

As used herein, an “array” comprises a support, preferably solid, with nucleic acid probes attached to said support. Arrays typically comprise a plurality of different nucleic acid probes that are coupled to a surface of a substrate such that the sequence and position of each member of the array is known. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., Science, 251:767-777 (1991). These arrays may generally be produced using mechanical synthesis methods or light directed synthesis methods that incorporate a combination of photolithographic methods and solid phase synthesis methods. Techniques for the synthesis of these arrays using mechanical synthesis methods are described in, e.g., U.S. Pat. Nos. 5,384,261, and 6,040,193. Although a planar array surface is preferred, the array may be fabricated on a surface of virtually any shape or even a multiplicity of surfaces. Arrays may be nucleic acids on beads, gels, polymeric surfaces, fibers such as fiber optics, glass or any other appropriate substrate. (See U.S. Pat. Nos. 5,770,358, 5,789,162, 5,708,153, 6,040,193 and 5,800,992.)

Preferred arrays are commercially available from Affymetrix Inc. and Agilent Technologies and are directed to a variety of purposes. (See Affymetrix Inc., Santa Clara and its website at www.affymetrix.com; Agilent Technologies, Santa Clara and its website at www.chem.agilent.com.)

As used herein “genomic nucleic acid molecule” refers to a DNA comprising or consisting of a segment of nucleic acid sequence identical to a segment of nucleic acid sequence found in a source genome. Thus, a vector having a recombinantly introduced segment of nucleic acid sequence found in a source genome (e.g., BAC or YAC) would also be considered a genomic nucleic acid molecule. Similarly, a cDNA molecule would also be considered a genomic nucleic acid molecule. Thus, “genomic nucleic acid molecule” is not limited to molecules directly from a genome but also includes molecules that are derived from a genome and contain genomic sequence information, as is understood by one skilled in the art.

As used herein “genomic nucleic acid fragment” refers to a genomic nucleic acid molecule or a fragment thereof. Fragments of genomic nucleic acid molecules can be prepared in a nonspecific manner (for example, random shearing), or in a specific manner (for example, using a restriction enzyme).

As used herein “source genome” is used herein to refer to all or a portion of the genomic nucleic acid sequences of an organism.

As used herein “genomic element” includes fixed, non-fixed and multicopy nucleic acid sequences having a defined sequence or a sequence substantially homologous to a defined sequence to a degree sufficient to permit hybridization with a targeting element under the hybridization conditions employed. Genomic elements of interest in the context of the present invention are found within a genomic nucleic acid fragment.

As used herein “multicopy nucleic acid” and “repeated genomic element” refer to nucleic acid sequences that are identical or that share a very high homology with each other, such as, for example, at least 80%, 81%, 82%, 83%, 84%, 85%, 86%, 87%, 88%, 89%, 90%, 91%, 92%, 93%, 94%, 95%, 96%, 97%, 98% or 99% homology and that are found in the same genome.

As used herein “targeting element” refers to a molecule that binds or associates specifically to a nucleic acid sequence in a population of nucleic acid molecules. In some embodiments, the targeting element is a nucleic acid, or nucleic acid derivative that hybridizes to a complementary target sequence in a population of nucleic acids. Examples of nucleic acid-based nucleic acid derivatives include, e.g., an oligonucleotide, oligo-peptide nucleic acid (PNA), oligo-LNA, or a ribozyme. The targeting element can alternatively be a polypeptide or polypeptide complex that binds specifically to a target sequence. Examples of polypeptide-based target elements include, e.g., a restriction enzyme, a transcription factor, RecA, nuclease, or any sequence-specific DNA-binding protein. The targeting element can alternatively or in addition be a hybrid, complex or tethered combination of one or more of these targeting elements.

Association of a targeting element with a sequence of interest can occur as part of a discrete chemical or physical association. For example, association can occur as part of an enzymatic reaction, chemical reaction, physical association; polymerization, ligation, restriction cutting, cleavage, hybridization, recombination, crosslinking, or pH-based cleavage. In a preferred embodiment, the targeting element is a nucleic acid of defined sequence and sufficient complementarity and length to permit selective hybridization with at least a portion of a genomic element of interest. Targeting elements employed in the present invention may already have an associated separation group prior to hybridizing with a genomic DNA fragment.

As used herein, “flanking element” refers to a nucleic acid sequence adjacent to a genomic element of interest in a genomic DNA fragment.

As used herein, “location” in a genome or in a sample of genomic nucleic acid molecules refers to the approximate location within a genome for a genomic element particularly a non-fixed genomic element that can be identified using the methods of the present invention. As will be appreciated by the skilled artisan, the degree of proximity of a flanking nucleic acid sequence identified by a method of the present invention to a genomic element of interest present in a genomic nucleic acid fragment is only as fine as the genomic sequences presented on a microarray. Thus, for example, if a 1 megabase genome is represented on a microarray by 10,000 evenly spaced oligonucleotides, each oligonucleotide 50 bases, the location of a genomic element within that genome can be determined to a specificity of at best 50 bases. In contrast, if a 10 megabase genome is represented on a microarray by the same number and length of oligonucleotides, the location of a genomic element within that genome can only be determined to a specificity of at best 500 bases. As will further be appreciated by the skilled artisan, a finer resolution in the latter case could be obtained by using multiple microarrays (for example, 10 microarrays each corresponding to a 1 megabase portion of the 10 megabase genome) or by increasing the density of spots on the microarray. A higher resolution can be obtained by using the invention in the embodiment where the captured genomic nucleic acid fragments of interest are immobilized on a surface and labeled through the generation of primer extension products using the isolated genomic nucleic acid fragment from the first step as template, thereby determining the sequence of the captured fragments.

As used herein, “separation group” refers to any moiety that is capable of facilitating isolation and separation of an attached targeting element that is itself associated with a genomic DNA fragment. Preferred separation groups are those which can interact specifically with a cognate ligand. A preferred separation group is an immobilizable nucleotide, e.g., a biotinylated nucleotide or oligonucleotide. Other examples of separation groups include ligands, receptors, antibodies, haptens, enzymes, chemical groups recognizable by antibodies or aptamers. A separation group can be immobilized on any desired substrate. Examples of desired substrates include particles, beads, magnetic beads, optically trapped beads, microtiterplates, glass slides, papers, test strips, gels, other matrices, nitrocellulose, nylon. The substrate includes any binding partner capable of binding or crosslinking with a separation group associated in a complex with a targeting element and a genomic DNA fragment. For example, when the separation group is biotin, the substrate can include streptavidin.

As used herein, “probe” refers to a polynucleotide having sufficient length to specifically hybridize under the hybridization conditions employed to an oligonucleotide or polynucleotide having a complementary nucleic acid sequence which is immobilized on an array. A probe is referred to as a “labeled probe” if the probe is covalently associated with a compound and/or element that can be detected due to its specific functional properties and/or chemical characteristics, the use of which allows the probe to which it is attached to be detected, and/or further quantified if desired, such as, e.g., an enzyme, an antibody, a linker, a radioisotope, an electron dense particle, a magnetic particle and/or a chromophore or combinations thereof, e.g., fluorescence resonance energy transfer (FRET). There are many types of detectable labels, including fluorescent labels, which are easily handled, inexpensive and nontoxic.

As used herein, “amplification” refers to an increase in the amount of nucleic acid sequence, wherein the increased sequence is the same as or complementary to the pre-existing nucleic acid template. Linear amplification excludes use of PCR amplification. Linear amplification is a method of geometric increase in copy number rather than an exponential increase in copy number. Amplification as used herein can also include the use of multiple labeled nucleotides during primer extension reactions in a sequence-dependent incorporation.

### Discussion of Specific Embodiments

In one embodiment, the method of the invention is divided into seven steps.

1) Providing a Population of Genomic Nucleic Acid Fragments

In this step, genomic nucleic acid molecules are extracted from cells of interest, using any number of standard protocols or kits. In general, genomic nucleic acid molecules from two or more source genomes are obtained in order to permit comparison of source genomes, but DNA from one source alone can also be used and compared against a previously established pattern of hybridization. Usually the source is a clonal population of cells, but can be any source, including mixed populations such as tissues, as well as tissue culture cells, colonies grown in liquid media, etc. This genomic DNA can potentially be used without further modification or may be digested with appropriate restriction enzymes or sonicated to appropriate random sizes. Factors governing the appropriate size of genomic DNA fragments depend on the frequency and size of the genomic element of interest as well as the size of the genome, and the density of the array. Genomic DNA may be reduced in length by enzymatic digestion with appropriately determined restriction enzymes, depending on the application. Alternatively, the long genomic DNA can be mechanically and randomly sheared to a desired length. In other situations, any shearing that may occur unavoidably in Step 1 may be sufficient to reduce the chromosomal DNA to a length usable in this invention, although the final length of DNA may vary depending on the particular application. Fragmentation of the DNA such as by shearing or enzymatic digestion may also be carried out after the extraction step, but before its immobilization on the surface or microarray.

2) Contacting said Population of Genomic DNA Fragments with a First Targeting Element

One or more targeting elements are made based on the specific application and genomic element. The targeting element may be one or more of those discussed above. The targeting element can itself be covalently attached or topologically linked to the targeted polynucleotide, which allows washing steps to be performed at very high stringency that result in reduced background and increased specificity.

In one preferred embodiment, the targeting element is an oligonucleotide that hybridizes to the nonfixed or multicopy genomic sequence. In such embodiments, the general considerations for targeting element sequence selection are as follows:

A) Since the purpose of the targeting element is to hybridize to genomic DNA fragments that contain the genomic element of interest, along with unique flanking elements, and since this DNA is generally double stranded, non-overlapping probes complementary to both strands of the genomic element of interest are typically generated.

B) Since unique flanking element information on both sides of the genomic element of interest are usually valuable, probes can be made near the 5′ and 3′ end of the genomic element of interest, particularly if the genomic element of interest is long (i.e. more than 1-5 kb). These 5′ and 3′ probes can be pooled or used separately depending on the specific application.

Targeting elements can target individual (unique) sequence elements, such as breakpoints, to determine their surrounding sequence context and linkage to other genomic sequences, or they can be designed to target several types of sequence elements simultaneously, such as both Ty1 and Ty2, or other classes of repeated elements, for their separation into subpopulations that have a reduced complexity compared to the original sample.

In a preferred embodiment, one or more probes are combined with the genomic DNA fragments from step 1 in the presence of Qiagen HaploPrep Hybridization Buffer (Cat. # 4310001) and the DNA is heat denatured and then reannealed.

Targeting elements may already have an attached separation group or a separation group can be added before proceeding to the third step. For example, a templated enzymatic extension step can be used to specifically attach biotinylated nucleotides only to those DNA sequences that result in complete hybridization of targeting elements, but not to other genomic DNA fragments.

For example, in preferred embodiments, the targeting element is an oligonucleotide with an extendable 3′ hydroxyl terminus and the separation group is an immobilizable nucleotide (such as a biotinylated nucleotide). In these embodiments, the separation group is preferably attached to the targeting element by extending the oligonucleotide with a polymerase in the presence of the biotinylated nucleotide, thereby forming an extended oligonucleotide primer containing the immobilizable nucleotide. Further details on a templated enzymatic extension step and its use can be found in US Patent App. No. 2001/0031467, published Oct. 18, 2001.

Multiplexing may also occur. If desired, the method can be used with second, third, or fourth or additional targeting elements, each targeting element either for targeting a different nonfixed genomic element or each targeting element containing different information from the others to allow binding of more than one targeting element to the same nonfixed genomic element, for example by use of oligonucleotides as targeting elements that bind at different sites to a transposon because they have different sequences. Multiplexing can occur by contacting the population of genomic nucleic acid fragments with an additional targeting element (e.g., a second, third, fourth or more targeting element) that binds specifically to an additional nucleic acid sequence or sequences of interest in the population of genomic nucleic acid fragments (which may be the same or different than the first nucleic acid of interest). A second (or additional) separation group is attached to the second targeting element. The attached second (or additional) separation group is attached to a substrate, thereby forming a second immobilized targeting element-separation group complex. The second separation group may be the same or different from the first separation group. The immobilized targeting element-genomic nucleic acid fragment complex is then removed from the population of genomic nucleic acid fragments, thereby separating the nucleic acid fragment of interest from the population of genomic nucleic acid fragments. A kit containing reagents useful for multiplexing, particularly by increasing the number of different targeting elements to target the same nonfixed genomic element, is also within the scope of the present invention.

Targeting elements can be also used in successive extractions by repeating an extraction using either the same or different targeting elements, thus targeting either the same or different sequence elements of interest in subsequent reactions. The purpose of successive isolations is to increase the specificity of the resulting overall isolated genomic material. For example, it is possible to use primer sets as targeting elements that have been designed for PCR. The advantage is that the forward and reverse primers provide multiplicative selectivity in targeting approximately the same locus or region by using two different targeting elements. Any cross-reactivities that may occur with respect to the first primer can be avoided in the second isolation round, where a different sequence of the same overall region is targeted by the second primer.

If the genomic nucleic acid fragments are in double-stranded form, the targeted location has to be rendered accessible in order for a targeting element (if an oligonucleotide) to bind to the fragment. This can be accomplished by heating the sample to a temperature at which the DNA begins to melt and form loops of single-stranded DNA. For example, the DNA may be heated to 90-95° C. for two to ten minutes. Alternatively, alkaline denaturation may be used. Under annealing conditions and typically in an excess of targeting oligonucleotide relative to template, the oligonucleotides will—due to mass action as well as their usually smaller size and thus higher diffusion coefficient—bind to homologous regions before renaturation of the melted genomic nucleic acid fragment strands occurs. Oligonucleotides are also able to enter double-stranded fragments at homologous locations under physiological conditions (37° C.). Methods and kits have been developed to facilitate the sequence-specific introduction of oligonucleotides into double-stranded targets such as genomic or plasmid DNA. A coating of oligonucleotides with DNA-binding proteins such RecA (E. coli recombination protein “A”) or staphylococcal nuclease speeds up their incorporation several orders of magnitude compared to the introduction of analogous unmodified oligonucleotides at higher concentration and significantly increases the stability of such complexes, while still permitting enzymatic elongation of the introduced oligonucleotide.

3) Immobilizing said Attached Separation Group to a Substrate

In the third step, targeting elements are immobilized by their attached separation group to a substrate. The method of immobilization to a substrate depends on the nature of the separation group. Any suitable method of immobilization of a nucleic acid molecule complex may be used. In a preferred embodiment, the separation groups are biotinylated nucleotides and the substrate consists of commercially available magnetic beads coated with streptavidin.

4) Separating Immobilized Genomic DNA Fragments from Non-Immobilized Genomic DNA Fragments

The method of separation depends on the method of immobilization. In a preferred embodiment, specific genomic DNA fragments containing the genomic elements of interest and their flanking elements are fixed to the magnetic beads by way of association with a targeting element having a biotinylated separation group while all other DNA is removed by a series of high stringency wash steps. After several wash steps, the bound DNA is released from the beads by heating in Qiagen EB buffer (included in HaploPrep Cartridge Cat. # 4340001/H100.C48) or in deionized water.

5) Preparing Labeled Probes

In this step, labeled probes having nucleic acid sequences complementary to sequences present in flanking elements are prepared. Probes may be labeled using any label known to those skilled in the art that will allow detection of hybridization to an array and not interfere with that hybridization. Fluorescent labeling is preferred. In one embodiment, after isolation of genomic nucleic acid fragments from the genomic nucleic acid fragment population, the isolated fragment is linearly amplified to ensure sufficient amounts of nucleic acid for hybridization to the microarray. In one embodiment, linear amplification of less than 100 fold is used. Labeling can optionally include multiple, distinguishable labels for different bases of the template that permit the determination of the sequence of the labeled probes. The labeling can occur and also be directly observed on a single molecule basis, such as by primer extension on a surface by using the immobilized genomic nucleic acid fragments as a template, thereby determining the sequence of the captured fragments. See, for example, WO2006084132.

During or after such amplification, a label is applied to the nucleic acid. Such amplification may be avoided if the population of fragments is sufficiently large and/or the nonfixed element is present in sufficient copies, as the skilled artisan can readily appreciate.

6) Applying said Labeled Probes to a Microarray

In general, labeled probes are combined with commercially available hybridization buffer, heated to separate DNA strands and applied to a microarray slide. Microarray slides may be from commercially available sources (e.g. Agilent, Affymetrix, etc) or home made. Each spot on the slide may consist of single stranded oligonucleotides, denatured PCR products, plasmids, BACs, YACs, or other distinguishable sources of DNA. In certain cases, hybridization of labeled DNA to repetitive DNA sequences present on the array spots will need to be masked by pre- or co-hybridization with unlabelled Cot-1 DNA from the species of interest. If labeling occurs as part of a sequencing reaction, the captured genomic fragments of interest can be ligated to a generic linker, such as to poly-dT or -dA tails. This linker serves to anchor the fragments to the surface by hybridization to randomly present, complementary poly-dA or -dT oligos that have been immobilized on the surface. The immobilized oligos can then serve as primers to initiate fluorescent, sequence-dependent labeling using the captured fragments as template.

The factors governing hybridization of labeled probes to a micro-array, including the length of the labeled probes, the length of the oligonucleotides immobilized on the micro-array, and the hybridization conditions, are well known in the art. In general, various degrees of stringency of hybridization may be employed. As the conditions for hybridization become more stringent, there must be a greater degree of complementarity between the labeled probe and an oligonucleotide immobilized on the micro-array for duplex formation to occur. The degree of stringency may be controlled by temperature, ionic strength, pH and/or the presence of a partially denaturing solvent such as formamide. For example, the stringency of hybridization is conveniently varied by changing the polarity of the reactant solution through manipulation of the concentration of formamide within the range of 0% to 50%. The degree of complementarity (sequence identity) required for detectable binding will vary in accordance with the stringency of the hybridization medium and/or wash medium. For purposes of the methods of the present invention, hybridization conditions are preferably optimized such that the degree of complementarity required for binding of labeled probe approaches 100 percent.

High stringency conditions for nucleic acid hybridization are well known in the art. For example, conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleotide content of the target sequence(s), the charge composition of the nucleic acid(s), and to the presence or concentration of formamide, tetramethylammonium chloride or other solvent(s) in a hybridization mixture.

7) Detecting Bound Labeled Probes

After hybridization, slides are washed according to standard protocols to remove unbound or poorly bound labeled probes from oligonucleotides immobilized on the microarray; the slides are dried and then read using a commercially available microarray scanner. Aside from a background level of annealing, the majority of hybridization to oligonucleotides (or polynucleotides) immobilized on the microarray will occur at locations representing the genomic element of interest as well as the sequences flanking the genomic element of interest, up until the nearest restriction site or to the site of random shearing (depending on how the genomic DNA fragments were prepared) flanking the genomic element of interest. Since the chromosomal coordinates of each oligonucleotide (or polynucleotide) on the array are known, a hybridization signal indicates that the element of interest is present in that vicinity in the original genome. For elements of interest in which their chromosomal positions are multiple and variable in different strains or individuals (e.g. active transposable elements or individual clones from a transposon library), the hybridization data will be most useful as a ratio of relative signal intensity for the two differentially labeled sources. A typical ratio analysis for S. cerevisiae chromosome V is shown in the figures and the following examples, in which both strains are isogenic except that strain FY2 contains 1 additional retrotransposon Ty1 element inserted within the URA3 gene. The green peak represents a high ratio of hybridization to the spots covering and flanking URA3 in strain FY2, compared to isogenic FY5 which lacks a Ty1 element at this location. The method of detecting bound labeled probes depends entirely on the nature of the label associated with the probe. Regardless of the type of label used, acquisition of data can involve the use of commercially available microarray scanners and software.

In another embodiment, the sequence information on a flanking element identified using the above seven method steps is used for the additional step of preparing a suitable PCR primer, which in conjunction with a primer specific to the genomic element of interest (or another flanking sequence specific primer), followed by PCR amplification and sequencing, to identify the precise genomic location of the genomic element of interest.

### EXPERIMENTAL SECTION

### Example 1

### Introduction

The model eukaryote S. cerevisiae has been at the forefront of studies of retrotransposons, i.e. transposons that use reverse transcriptase for their replication, and which copy and paste themselves to new genomic locations. Several distinct families of retrotransposons, or “Tys” have been identified in this organism, both anecdotally, and systematically through the genome sequencing effort. In the only fully sequenced S. cerevisiae strain, S288c, the most abundant transposons are Ty1 (31 copies) and Ty2 (11 copies). These closely related 5.9 kb full-length mobile elements consist of two overlapping open reading frames, each of which encodes several proteins. The coding regions are flanked by ˜300 bp nearly identical long terminal repeats (LTRs). Ty4 (3 copies) is a distinct and less abundant element with a similar structure. Ty3 (2 copies) is another distinct element, with a different arrangement of protein coding segments, but still with flanking LTRs. Ty5 is only a vestigial element, with no intact copies in the S. cerevisiae genome (Kim et al., 1998). The insertion site preferences of these different families is characteristic, with most Ty1 and Ty2s, and all Ty3 and Ty4 elements found near to tRNA sequences (Voytas and Boeke, 1993), and Ty5 fragments found within silenced DNA [Zou, 1996 #2800]. For each full length Ty element there are an order of magnitude more solo LTR elements dispersed through the genome. These are thought to have arisen by LTR-LTR recombination of full-length elements, with looping out of the internal regions.

The complete sequence of strain S288c provides a snapshot of retrotransposon positions in one S. cerevisiae strain at one point in time (Goffeau et al., 1996). But transposons are dynamic, and strain-specific new insertions, recombinational losses, and potential rearrangements will likely result in a much more complex picture of genome interaction than can be gleaned from a single complete genome sequence. In the absence of complete sequencing of many different clones and strains, we have developed a way to identify the location of transposons in a genome and compare their organization with those in other strains or individuals.

Material and Methods:

Strains and DNA: All strains used were obtained from the Botstein Lab Collection, and included FY2, FY3 and FY5 (all derivatives of S288c), RM11-1a (Brem et al., 2002; Yvert et al., 2003), Cen.PK (Entian et al., 1999), W303 (Rothstein, 1983; Thomas and Rothstein, 1989), and SKI (Kane and Roth, 1974; Kelly et al., 1983). Genomic DNA was obtained by growing up 100 ml cultures in YPD and then purifying DNA using Qiagen Genomic DNA buffer Set™ and Genomic-tip 500/G™. Purified DNA was stored frozen in water. Two-three micrograms of DNA were separately digested with AflII, EcoRI, or SphI (New England Biolabs) as per manufacturer's instructions, then precipitated and resuspended in ddH₂O. Equal volumes of differently digested DNA were pooled for subsequent extraction.

Transposon Specific Extraction (TSE): ˜500 ng of pooled digested DNA was mixed with one or more oligonucleotide primers (referred to as “probe”) in a buffer containing dNTPs, one of which has an attached biotin group, and with Qiagen HaploPrep Hybridization Buffer (Cat. # 4310001) which contains a thermostable DNA polymerase. Probes can be made for yeast transposons and for the URA3 gene by selecting appropriate probes from the sequences identified at Genbank Accession Nos. M18706 (Ty1); X03840 (Ty2); M23367 (Ty3); X67284 (Ty4); and K02207 (URA3). For example, a set of probes were designed to selectively capture both Ty1 and Ty2 elements in yeast. A CLUSTAL sequence alignment of all 39 Ty1 and Ty2 elements was used to identify regions that are conserved between the two types. The complete elements are about 5900 bases long. However the first and last 340 base pairs were not considered for selecting probe locations since they represent long terminal repeats (LTRs) that are also present, by themselves, in about 300 other places in the genome. The positions in the first two probes (391-1 and 491-2) were chosen to target the 5′-end of the transposon in generally conserved regions within the following locations (*=Ty1/Ty2-consensus sequence):

| YBLWTy1-1 | GTAGCGCCTGTGCTTCGGTTACTTCTAAAGAAGTC | 393 | (targeted strand) | (SEQ ID NO: 1) |
|---|---|---|---|---|
| | ******** ****************** ****** | | | |
| YBLWTy1-1 | ACAACACCTCCCTCATCTGCTGTTCCAGAGAACCA | 493 | | (SEQ ID NO: 2) |
| | ********* **** **************** | | | |
| The third probe (5046-2) was designed to target the transposon near its 3′-end: | | | | |
| YBLWTy1-1 | CTATTATACTACATCAACACACTTGCACAACATAT | 5003 | | (SEQ ID NO: 3) |
| | ** ********************** ******** | | | |

In all cases here the probe sequence corresponds to the forward/sense strand, and it is therefore targeting (binding to) the antisense strand of the captured template.

In a second round of experiments, after evidence that the probes appear to be pulling out preferentially one strand (i.e. the directly targeted one) over the other, the three forward-oriented probes were then complemented by the following three probes of reverse orientation (i.e. binding to the sense strand of the template; 460-1RC, 491-1RC and 5100-1RC):

| | | | | |
|---|---|---|---|---|
| YBLWTy1-1RC | TTTGGAAGCTGAAATGTCTAACGGATCTTGAGTTG | 393 | (3′-5′; = targeted strand) | (SEQ ID NO: 4) |
| | ** ********** * ************** ** | | | |
| (YBLWTy1-1 | CAACTCAAGATCCGTTAGACATTTCAGCTTCCAAA | 393) | | (SEQ ID NO: 5) |
| | ** ************** * ************* | | | |
| YBLWTy1-1RC | GAGGCATGATGATGGTTCTCTGGAACAGCAGATGA | 493 | (3′-5′; = targeted strand) | (SEQ ID NO: 6) |
| | *** ******* **************** **** | | | |
| (YBLWTy1-1 | TCATCTGCTGTTCCAGAGAACCATCATCATGCCTC | 493) | | (SEQ ID NO: 7) |
| | **** **************** ******* *** | | | |
| YBLWTy1-1RC | TTGGACGGAAATAGTATATGTTGTGCAAGTGTGTT | 5003 | (3′-5′; = targeted strand) | (SEQ ID NO: 8) |
| | * ** ** ************** *********** | | | |
| (YBLWTyL-1 | AACACACTTGCACAACATATACTATTTCCGTCCAA | 5003) | | (SEQ ID NO: 9) |
| | *********** ************** ** ** * | | | |

Note that probe 491-1RC is essentially complementary to 491-2 (forward orientation), and would therefore normally not be used together in one multiplexed TSE assay.

The mixture was heat denatured for 15 minutes at 95° C., then transferred to a Genovision Geno M™-6 robot, and allowed to renature and extend for 20 minutes at 65° C. Streptavidin-coated magnetic beads were then added to the mixture to capture the DNA attached to the biotin-containing extended probes. After several high stringency wash steps, the bound DNA is released from the beads by heating to 80° C. in Qiagen EB buffer. The supernatant is collected for fluorescent labeling. All reagents and buffers, starting with the streptavidin-coated magnetic beads are included in Genovision HaploPrep Cartridge (Cat. # 4340001/H100.C48), used in conjunction with the robot.

Microarray Procedures: Because recovery of DNA by the TSE procedure is not quantitative and the amount of extracted DNA is below simple detection, a volume of 10.5 microliters were mixed with 10 ul 2.5× random primer mix (Invitrogen), and labeling was performed using Cy3 or Cy5 liganded dUTP or dCTP as per the Invitrogen BioPrime CGH labeling kit, which uses exo-Klenow fragment of E. coli DNA polymerase to extend from the random primers and add the fluorescently labeled nucleotide. The products of the polymerization reaction were purified through Zymo Research DNA Clean and Concentrator spin column (catalog #D4003), resuspended in ddH₂O, and the quantity and incorporation of dye were measured using a Nanoprop® ND-1000 Spectrophotometer. Comparative genomic hybridization (CGH) was then performed using either Agilent Yeast V2 Oligo Microarrays (Cat. # G4140B and referred to as “ORF arrays”) or Yeast Whole Genome (1 design) ChIP-on chip microarrays (Cat. # G4486A and referred to as “chip arrays”). In the former case 250 ng of each sample were combined, mixed with control fragments, heated to 95° C. and then mixed with 2× hybridization buffer (Agilent) before adding it to the microarray slide. In the latter case, a similar procedure was used except 500 ng of each sample was used. Hybridization was carried out at 60° C. for 17 hours. Slides were washed according to manufacturer's instructions, dried in acetonitrile and then scanned using an an Agilent Microarray Scanner. Intensity data was obtained using Agilent Feature Extractor. Feature extracted information, including log2 ratio of Cy3 and Cy5 signal in each spot, as well as the mean intensity in each spot for each color was used to determine the location of sequences flanking transposons. Data from the arrays were graphically expressed using Java TreeView.

Affymetrix Arrays Biotinylated probes for Affymetrix tiling arrays were made according to published procedures (Gresham et al., 2006), and location of polymorphic sites along each chromosome was determined using appropriate software.

PCR and sequencing procedures: Confirming PCR primers were designed using Primer3, and PCR products were obtained by standard means using Taq polymerase (Roche). Certain products were purified through Zymo columns and sequenced by Genewiz™ using one of the PCR primers as the sequencing primer.

Results:

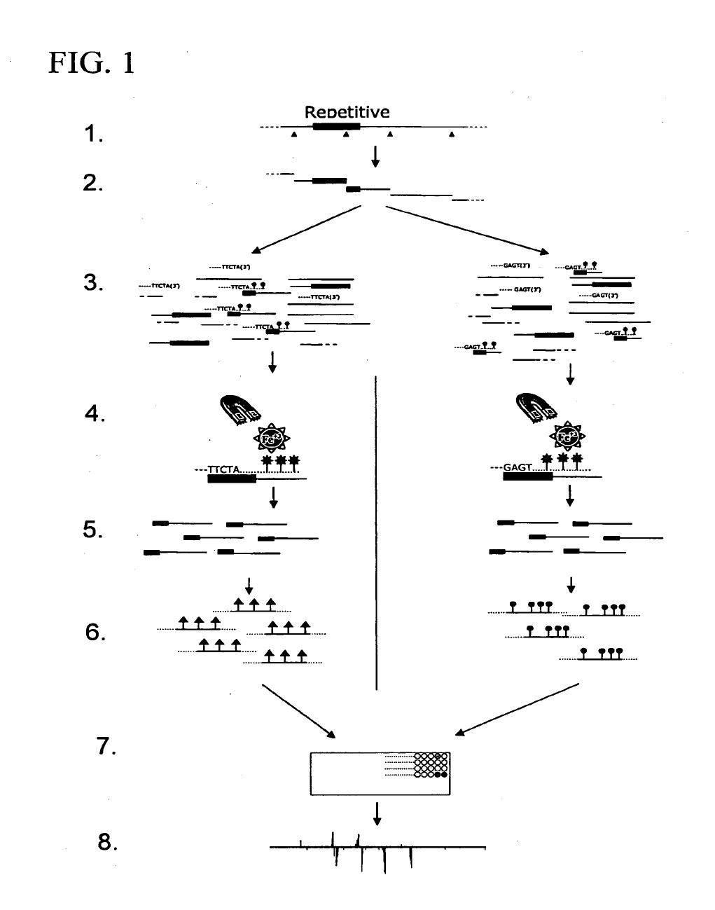

Description of General Method: Our method is based on the principle that while specific members of a family of transposons tend to be highly similar, the flanking sequence into which different members are inserted is likely to be unique. Therefore identification of these flanking sequences will reveal the location of the adjoining transposons. In order to isolate DNA fragments containing sequences that flank specific transposons, we digested whole genomic DNA with three different restriction endonucleases, pooled the digested DNA, and combined it with one or more oligonucleotide primer designed to anneal to specific segments of selected transposons (FIG. 1, steps 1-3). Incubation of the DNA and oligonucleotides in the presence of a DNA polymerase and nucleoside triphosphates, one of which is biotinylated, results in the addition of biotinylated bases to the extended primer. Subsequently, magnetic beads coated with streptavidin are added and used to separate the annealed fragments from all other genomic DNA fragments (FIG. 1, steps 3-4). The extracted DNA fragments are released from the beads and fluorescently labeled using either Cy3 or Cy5 dUTP (or dCTP) in the presence of random primers and exo-Kienow fragment (FIG. 1, step 5), and then hybridized to dense whole genome oligonucleotide microarrays of the S. cerevisiae genome (FIG. 1, steps 6-7). We took advantage of the power of comparative hybridization to minimize noise from non-specific or fortuitous extraction fragments, and to accentuate differences in extracted fragments from two different sources of genomic DNA or from extraction of the same genomic DNA using oligonucleotide primers specific to different transposons.

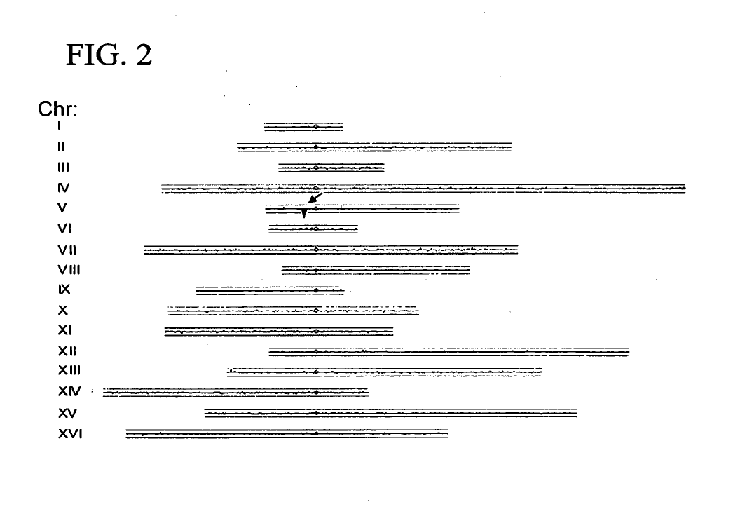

Comparison of isogenic strains: We first used this method to identify one new Ty1 insertion in otherwise isogenic strains containing ˜40 Ty1 and Ty2 elements. FY2 and FY5 are isogenic derivatives of S288c, differing only by the presence of a full-length Ty1 element in the URA3 gene of FY2. We annealed digested DNA from either strain with a pool of 5 probes corresponding to internal sequences common to both Ty1 and Ty2. Analysis of the log₂ratio of normalized intensity per spot on an Agilent array showed near perfect agreement between the two strains, aside from a significant difference in hybridization intensity on the left arm of chromosome 5 (FIG. 2). The peak difference, spanning a distance of ˜8 kb correspond roughly to the location of the nearest flanking cleavage sites for the restriction endonucleases initially used to digest the two DNAs. This result demonstrates that the extraction and mapping method can identify the location of a single differential transposon insertion.

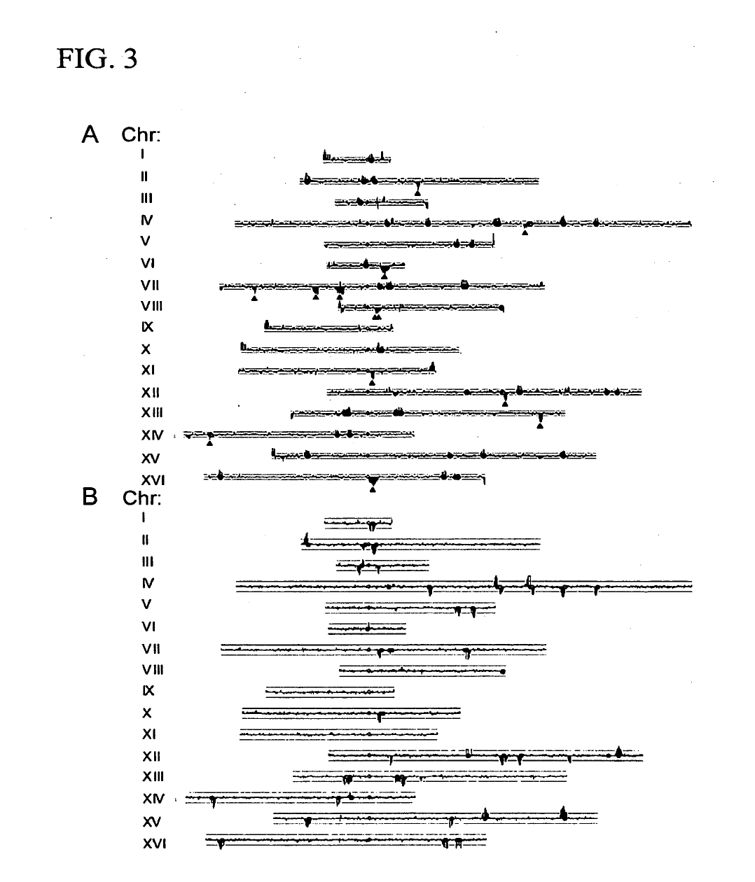

Comparison of two unrelated strains: We next validated the method by comparing the transposon content of two sequenced strains of S. cerevisiae: S288c and RM11. The S288c sequence comprised the first published eukaryotic genome, and the transposon content of this strain has been the subject of extensive analysis. RM11 was derived from a California vineyard, and was recently sequenced at the Broad Institute (www.broad.mit.edu/annotation/fgi/). Analysis of the two sequences has shown that they have no common full-length Ty1 or Ty2 elements (A. G., L. Kruglyak, and S. Pratt, in preparation). We used the same set of probes to extract both Ty1 and Ty2-associated fragments from either S288c or RM11 restriction endonuclease digested genomic DNA. We labeled the RM11 fragments with Cy3 (green) and the S288c fragments with Cy5 (red), and then hybridized the labeled DNA to an array. After washing and scanning, the relative hybridization intensity was calculated for each oligo feature on the array, and these values were aligned by position along each chromosome. We scanned the values on each chromosome and designated a location as a potential transposon peak if >5 consecutive features had log₂ratios of hybridization signal greater than 1.58, corresponding to a 3 fold difference in relative intensity of one dye over the other. Peaks located within 10 kb of one another were joined. These criteria were chosen to optimize the balance between false positives and false negatives. As shown in FIG. 3A, we observed 48 peaks for S288c and 23 peaks for RM11. Changing the cutoff value or the number of consecutive probes meeting the cutoff increased either the false positive rate or the false negative rate (data not shown).

While the arrays identified real transposon elements, there were also false positive peaks. These occurred primarily in telomeric and subtelomeric regions, and were variable depending on the strains used. Although we do not yet understand the basis for these false-positive peaks, they likely are related to the highly repetitive nature of the sequences near telomeres and the unequal distribution of subtelomeric X and Y′ elements in different strains.

More interestingly, four peaks from DNA derived from S288c were unannotated in SGD but also showed up on the Ty1 vs. Ty2 array. Two were present on the right arm of chromosome III, centered at ˜145000 and ˜169000. The official map of S288c shows several solo LTRs at these locations but no full-length Ty1 or Ty2 elements. We confirmed by sequence analysis that these two unannotated peaks are in fact Ty1 elements, and their organization is complex (data not shown). In particular two Ty1s are present at ˜169000, in a head to head orientation. Interestingly, the Tys on chromosome III have been previously described and their polymorphic distribution in different yeast strains studied (Lemoine et al., 2005; Warmington et al., 1987; Stucka et al., 1989; Wicksteed et al., 1994). Their existence is discussed in the original report of the complete chromosome III sequence (Oliver et al., 1992). Two other unexpected peaks were on chromosome XII, one centered at ˜219000 and the other at ˜816000. The former is listed in SGD as an ORF, but is annotated as a partial Ty1 element. The latter has a solo LTR and a tRNA listed in SGD, but no apparent Ty elements. We used combinations of PCR primers on either side of the peak positions, as well as primers internal to Ty1 and Ty2, to confirm the presence of the predicted Ty element, which is inserted at base 818,470, midway between the pre-existing LTR and tRNA at this location (data not shown).

### Example 2

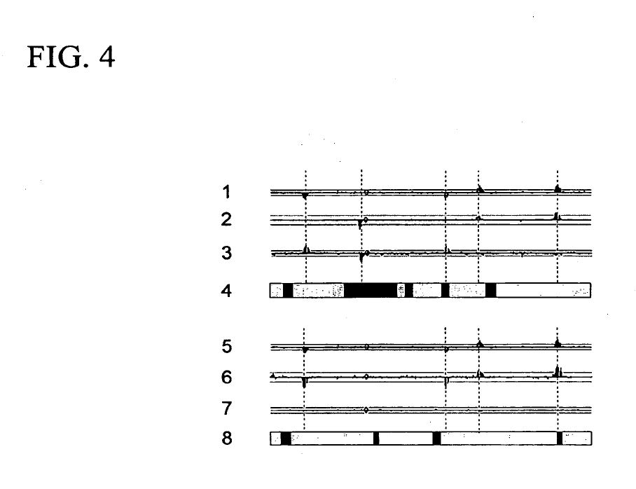

The following example shows how the method of the present invention can be used to extract and identify DNA associated with any specific sequence. In particular, probes were designed that would anneal to internal regions of Ty1 or Ty2, exploiting the regions of maximum differences between these two families of closely related elements. As shown in FIG. 4, when Ty1-associated fragments were labeled with Cy3 and Ty2-associated fragments with Cy5, each initial Ty1/2 peak could be correlated with the respective element associated with it. We extended this analysis to identify the three Ty3 full-length elements and Ty3 and Ty4 solo LTR elements in the S288c genome.

### Example 3

The following example shows how the method of the present invention can be extended to partially unmapped strains.

A comparison was made in the pattern of transposons in S288c with those in two common lab strains, CenPK and W303. In each of these cases, the strain was originally derived from a cross between S288c and an unrelated strain, although the detailed histories and origins are not completely documented. Previous work has shown that these strains are patchworks, with blocks of S288c sequence interspersed with blocks from the other parent (Daran-Lapujade et al., 2003; Winzeler et al., 2003). Using Affymetrix yeast tiling arrays, which are based on the S288c sequence, the patchwork nature of these strains is easily observable (FIG. 4), since SNPs are much more likely to be present and detected for segments derived from the non-S288c parent. We took advantage of this analysis to align each S288c, W303, and CenPK chromosome with the respective chromosome tracing derived from yeast tiling array. For chromosome 12, in most cases the strain of origin of the CenPK segment could explain the presence or absence of a transposon at a given locus. For 54 cases of peaks in S288c and/or CenPK, 24 corresponded to common transposons in regions of the CenPK genome derived from S288c. Similarly, in 17 cases where peaks were present in S288c or in CenPK, the corresponding portion of the CenPK genome was not derived from S288c. However, several anomalous cases were also observed. In one case, there were coincidental transposons in the same region of both strains, even though the portion of cenPK was not of S288c origin. In 4 cases, a Ty element was present in cenPK, but not in S288c, despite the finding that the insertion was likely in an S288c-derived region. Conversely, there were 2 cases where a Ty element was not present in CenPK, but would have been predicted to be there based on its presence in S288c and the fact that the respective portion of the CenPK genome is derived from S288c.

A similar situation was seen for W303. Based on tiling array data, a much greater percentage of the W303 genome is derived from S288c. For W303, certain transposons were present at the same locations as their S288c counterpart and that segment of the chromosome was likely derived from S288c, while other transposons were distinct in each strain, and corresponded to regions of non-S288c origin. Again there were ambiguous cases that will require further analysis to explain. These differences may be due to differences in transposon location in the specific S-288c parent strain that was used in the initial cross from which these hybrid strains were derived. However, an intriguing possibility for these aberrant events is that the process of mating and/or outcrossing results in mobilization of transposons in yeast, and we are observing the consequences of that mobility.

### Example 4

The following example shows how the methods of the present invention can be extended to completely unmapped strains. Specifically, the transposon content of SKI, a well known lab strain, unrelated to S288c was determined.

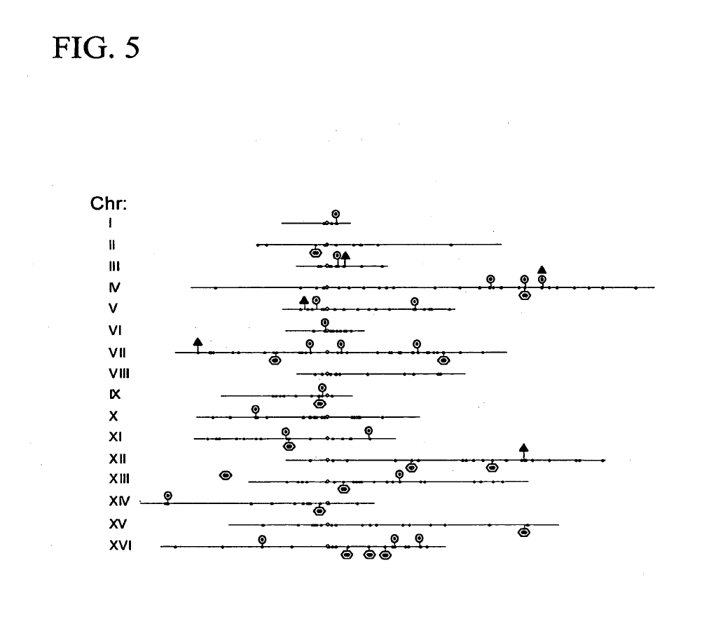

We next examined the transposon content of SKI, a commonly studied laboratory strain unrelated to S288c. Using a variety of transposon specific extraction probes we were able to identify 20 potential full-length Ty1 elements, 5 potential Ty2 elements, and 14 potential Ty3 LTRs. Based on these data, we generated the transposon map for SKI shown in FIG. 5 (the approximate coordinates of the insertions are given in Supplemental Table 2 referred to in Gabriel et al., 2006). In 94% of the predicted insertion sites, the peaks for the full-length element or LTR are closely linked to the known locations of tRNA genes, as expected from the known preferences of yeast retrotransposons. We confirmed our predicted placement for 7 Ty3 LTRs and 4 unique Ty1/Ty2 full-length elements, using a combination of PCR and sequencing. Thus our technique can quickly and accurately assign transposon locations in an otherwise unsequenced strain. In six cases the positions of transposons in S288c and SKI overlapped one another. Detailed sequence analysis will be required to determine whether these are the same evolutionarily conserved elements or different elements inserted in similar locations.

### Example 5

The following example shows how the methods of the present invention can be used to map artificial transposon insertions.

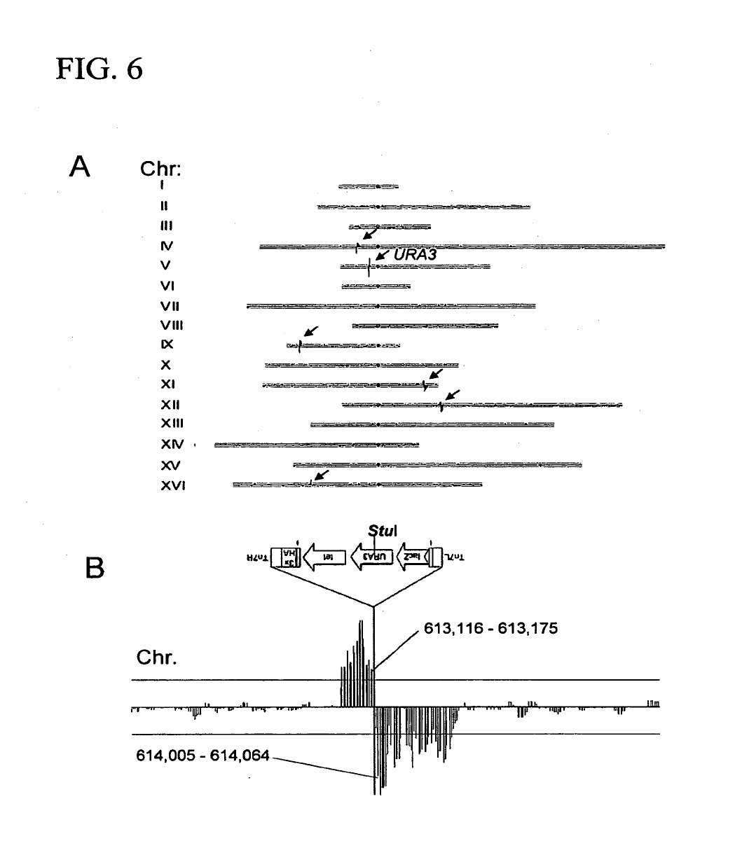

A number of methods have been described for genetic screens based on randomly inserting bacterial transposon sequences into plasmid-based yeast genomic libraries, and then transforming pools of the yeast DNA containing the bacterial transposons back into the yeast genome by recombination (Burns et al., 1994; Castano et al., 2003; Kumar et al., 2004; Merkulov and Boeke, 1998; Ross-MacDonald et al., 1997). This results in libraries of yeast clones each marked by a different bacterial insertional event, which can then be selected for phenotypically. To test our method for identifying the location of artificial transposon insertions in the yeast genome, we first sequenced the insertion junctions of five independent UR43 marked Tn7 based artificial transposons present in a plasmid-based yeast genomic library (Kumar et al., 2004). In this way we knew the precise insertion site for each artificial transposon. The yeast DNA segments from the five plasmids were transformed into yeast strain FY3 and cells that had acquired uracil prototrophy by homologous recombination of the segments were chosen. We then purified genomic DNA from the transformed strains, pooled the DNA, digested the pooled DNA with StuI and extracted fragments using probes specific to either the 5′ end or the 3′ end of URA3. We chose StuI because it cuts only once in the artificial transposon, in the center of the URA3 region. The extracted DNA samples were labeled with Cy3 (5′ flanking) or Cy5 (3′ flanking), and hybridized to an Agilent Whole Genome array. As shown in FIG. 6A, we observed 6 obvious regions of significant differential labeling (arrows), and these corresponded closely to the 5 sequenced insertion sites as well as URA3 itself on chromosome V. Thus our method can simultaneously identify multiple transposon insertions, each present in only a fraction of the population.

Thus, the methods of the present invention can be used to identify artificial as well as natural transposons whose location in the genome is not fixed.

### Example 6

The following example shows how the method of the present invention can be used to selectively isolate only one of multiple copies of a duplicated genomic region.

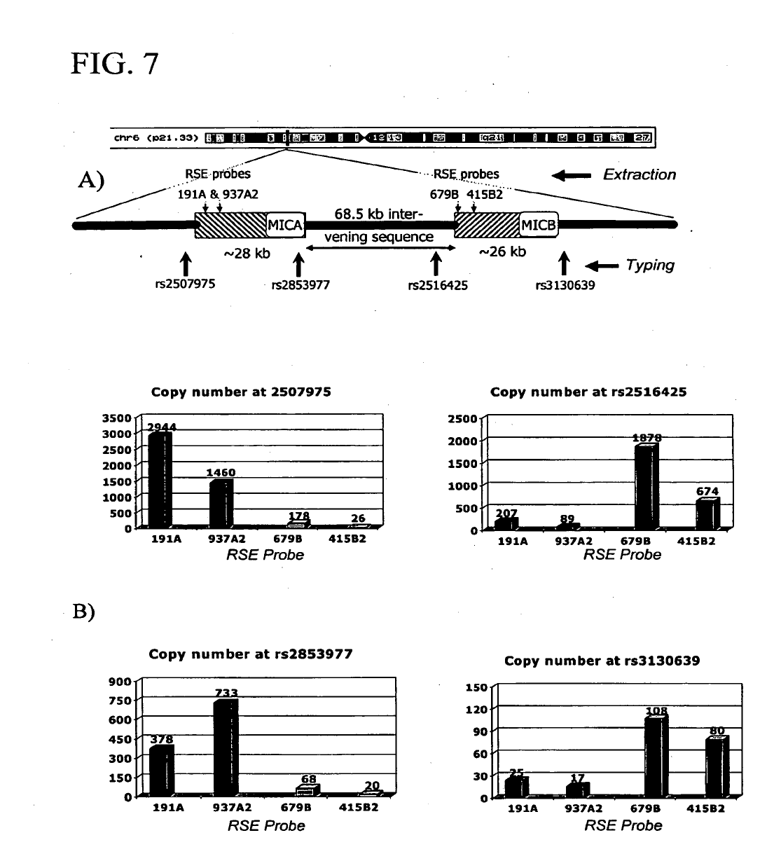

Four region specific extraction (“RSE”) probes were used to separately target specific polymorphisms in two highly homologous regions (93% identity) of the major histocompatibility complex (MHC) on chromosome 6 (FIG. 7). Specifically, RSE probes 191A & 937A2 target two sites in the MICA region, whereas RSE probes 679B 415B2 target two sites in the neighboring MICA region (FIG. 7a). MICA and MICB are divided by a 68 kb intervening sequence and due to their high degree of homology can be difficult to type separately in PCR or sequencing reactions. In this example, genomic DNA starting material of about 50 kb in length was used to target and isolate only the genomic region around one of the duplicated sequences (FIG. 7b). FIG. 7b shows copy number after typing by real time PCR.

By targeting a unique, single copy sequence element upstream or downstream of the targeted region, errors and ambiguities associated with the reconstruction of the duplicated region are thus avoided by separating the region of interest away from other material. The ability to capture known or unknown DNA sequences distal to the region of interest can be particularly useful to determine the location or orientation of sequence targets (such as repetitive elements or translocation breakpoints). This enables the analysis of linked regions that may only be partially known, such as deleted, inverted or otherwise structurally modified sequence elements, and determine their location, copy number and orientation.

Discussion

The above examples show that dense oligonucleotide microarrays are an efficient and accurate approach to identifying the location of polymorphic transposable elements throughout the yeast genome. By combining the power of comparative genomic hybridization to identify differences between two samples, with a robust and generalizable technique for sequence-specific DNA capture and purification, we have compared the transposon content of different strains, distinguished closely related Ty1 and Ty2 elements from the same strain, mapped the transposon locations of unknown strains, and identified artificial introns inserted into yeast strains as a genetic marker. The power of the technique comes from its ability to examine the whole genome simultaneously and provide positional information for further analysis. Previously, differences in Ty content of different strains has been reported anecdotally, but now we have the tools to get a complete picture of transposon positioning in any given yeast genome. This will have important implications in comparing phenotypic differences between different yeast strains, and for studying the evolutionary dynamics of transposons within the yeast genome.

There have been previous reports of using microarrays to identify the position of multiple artificial transposons inserted into genomes (Chan et al., 2005; Groh et al., 2005; Lawley et al., 2006; Mahalingam and Fedoroff, 2001; Salama et al., 2004; Tong et al., 2004), primarily in prokaryotes, but also in Arabidopsis. The present invention concerns using a microarray, and particularly array CGH, to identify the natural transposon population in a strain. A somewhat different approach to the same end has been submitted (S. Wheelan and J. Boeke, pers. Comm.) that uses vectorette PCR to pull out sequences from the yeast genome flanking transposons. In this regard it is notable that the method described here does not require ligation and or PCR amplification, and so is likely to be simpler, much less biased, and a more robust approach.

### REFERENCES

All of the references cited herein are hereby incorporated by reference herein in their entireties.
Brem, R. B., Yvert, G., Clinton, R., and Kruglyak, L. (2002). Genetic dissection of transcriptional regulation in budding yeast. Science 296, 752-755.
Burns, N., Grimwade, B., Ross-Macdonald, P. B., Choi, E., Finberg, K., Roeder, G. S., and Snyder, M. (1994). Large-scale analysis of gene expression, protein localization, and gene disruption in Saccharomyces cerevisiae. Genes and Dev 8, 1087-1105.
Castano, I., Kaur, R., Pan, S., Cregg, R., Penas Ade, L., Guo, N., Biery, M. C., Craig, N. L., and Cormack, B. P. (2003). Tn7-based genome-wide random insertional mutagenesis of Candida glabrata. Genome Res 13, 905-915.
Chan, K., Kim, C. C., and Falkow, S. (2005). Microarray-based detection of Salmonella enterica serovar Typhimurium transposon mutants that cannot survive in macrophages and mice. Infect Immun 73, 5438-5449.
Daran-Lapujade, P., Daran, J. M., Kotter, P., Petit, T., Piper, M. D., and Pronk, J. T. (2003). Comparative genotyping of the Saccharomyces cerevisiae laboratory strains S288C and CEN.PK113-7D using oligonucleotide microarrays. FEMS Yeast Res 4, 259-269.
Dunham, M. J., Badrane, H., Ferea, T., Adams, J., Brown, P. O., Rosenzweig, F., and Botstein, D. (2002). Characteristic genome rearrangements in experimental evolution of Saccharomyces cerevisiae. Proc Natl Acad Sci USA 99, 16144-16149.
Engels, W. R. (1996). P elements in Drosophila. Curr Top Microbiol Immunol 204, 103-123.
Entian, K. D., Schuster, T., Hegemann, J. H., Becher, D., Feldmann, H., Guldener, U., Gotz, R., Hansen, M., Hollenberg, C. P., Jansen, G., et al. (1999). Functional analysis of 150 deletion mutants in Saccharomyces cerevisiae by a systematic approach. Mol Gen Genet. 262, 683-702.
Gabriel, A., Dapprich, J., Kunkel, M., Gresham, D., Pratt, S., and Dunham, M. (December 2006). Global Mapping of Transposon Location. PIoS Genetics 2 (12), e212.
Goffeau, A., Barrell, B. G., Bussey, H., Davis, R. W., Dujon, B., Feldmann, H., Galibert, F., Hoheisel, J. D., Jacq, C., Johnston, M., et al. (1996). Life with 6000 genes. Science 274, 546, 563-547.
Gresham, D., Ruderfer, D. M., Pratt, S.C., Schacherer, J., Dunham, M. J., Botstein, D., and Kruglyak, L. (2006). Genome-wide detection of polymorphisms at nucleotide resolution with a single DNA microarray. Science 311, 1932-1936.
Groh, J. L., Luo, Q., Ballard, J. D., and Krumholz, L. R. (2005). A method adapting microarray technology for signature-tagged mutagenesis of Desulfovibrio desulfricans G20 and Shewanella oneidensis MR-1 in anaerobic sediment survival experiments. Appl Environ Microbiol 71, 7064-7074.
Han, J. S., Szak, S. T., and Boeke, J. D. (2004). Transcriptional disruption by the L1 retrotransposon and implications for mammalian transcriptomes. Nature 429, 268-274.
Kane, S. M., and Roth, R. (1974). Carbohydrate metabolism during ascospore development in yeast. J Bacteriol 118, 8-14.
Kazazian, H. H., Jr. (1998). Mobile elements and disease. Curr Opin Genet Dev 8, 343-350.
Kelly, S. L., Merrill, C., and Parry, J. M. (1983). Cyclic variations in sensitivity to X-irradiation during meiosis in Saccharomyces cerevisiae. Mol Gen Genet. 191, 314-318.
Kim, J. M., Vanguri, S., Boeke, J. D., Gabriel, A., and Voytas, D. F. (1998). Transposable elements and genome organization: a comprehensive survey of retrotransposons revealed by the Saccharomyces cerevisiae genome sequence. Genome Res 8, 464-478.
Kumar, A., Seringhaus, M., Biery, M. C., Samovsky, R. J., Umansky, L., Piccirillo, S., Heidtman, M., Cheung, K. H., Dobry, C. J., Gerstein, M. B., et al. (2004). Large-scale mutagenesis of the yeast genome using a Tn7-derived multipurpose transposon. Genome Res 14, 1975-1986.
Lawley, T. D., Chan, K., Thompson, L. J., Kim, C. C., Govoni, G. R., and Monack, D. M. (2006). Genome-wide screen for salmonella genes required for long-term systemic infection of the mouse. PLoS Pathog 2, e11.
Lemoine, F. J., Degtyareva, N. P., Lobachev, K., and Petes, T. D. (2005). Chromosomal translocations in yeast induced by low levels of DNA polymerase a model for chromosome fragile sites. Cell 120, 587-598.
Mahalingam, R., and Fedoroff, N. (2001). Screening insertion libraries for mutations in many genes simultaneously using DNA microarrays. Proc Natl Acad Sci USA 98, 7420-7425.
Merkulov, G. V., and Boeke, J. D. (1998). Libraries of green fluorescent protein fusions generated by transposition in vitro. Gene 222, 213-222.
Perez-Ortin, J. E., Querol, A., Puig, S., and Barrio, E. (2002). Molecular characterization of a chromosomal rearrangement involved in the adaptive evolution of yeast strains. Genome Res 12, 1533-1539.
Ross-MacDonald, P., Sheehan, A., Roeder, G. S., and Snyder, M. (1997). A multipurpose transposon system for analyzing protein production, localization, and function in Saccharomyces cerevisiae. Proc Natl Acad Sci USA 94, 190-195.
Rothstein, R. J. (1983). One-step gene disruption in yeast. Methods Enzymol 101, 202-211.
Salama, N. R., Shepherd, B., and Falkow, S. (2004). Global transposon mutagenesis and essential gene analysis of Helicobacter pylori. J Bacteriol 186, 7926-7935.
Scherer. S. W.; Green, E. D.; Human chromosome 7 (2004): A model for structural and functional studies of the human genome. Hum. Mol. Genet. 13 (October 1), Spec No 2:R303-313.
Shaw, C. J.; Lupski, J. R. (2004). Implications of human genome architecture for rearrangement-based disorders: the genomic basis of disease. Hum. Mol. Genet. 13 (April 1), Spec No. 1:R57-64.
Sorek, R., Ast, G., and Graur, D. (2002). Alu-containing exons are alternatively spliced. Genome Res 12, 1060-1067.
Spitz F, Herkenne C, Morris M A, Duboule D. (2005). Inversion-induced disruption of the Hoxd cluster leads to the partition of regulatory landscapes. Nat. Genet. 37(8), 889-93.
Stankiewicz, P.; Lupski, J. R. (2002). Molecular-evolutionary mechanisms for genomic disorders. Curr. Opin. Genet. Dev. 3 (June 12), 312-319.
Thomas, B. J., and Rothstein, R. (1989). Elevated recombination rates in transcriptionally active DNA. Cell 56, 619-630.
Tong, X., Campbell, J. W., Balazsi, G., Kay, K. A., Wanner, B. L., Gerdes, S. Y., and Oltvai, Z. N. (2004). Genome-scale identification of conditionally essential genes in E. coli by DNA microarrays. Biochem Biophys Res Commun 322, 347-354.
Voytas, D. F., and Boeke, J. D. (1993). Yeast retrotransposons and tRNAs. TIG 9, 421-427.
Winzeler, E. A., Castillo-Davis, C. I., Oshiro, G., Liang, D., Richards, D. R., Zhou, Y., and Hartl, D. L. (2003). Genetic diversity in yeast assessed with whole-genome oligonucleotide arrays. Genetics 163, 79-89.
Yu, X., and Gabriel, A. (2003). Ku-Dependent and Ku-Independent End-Joining Pathways Lead to Chromosomal Rearrangements During Double-Strand Break Repair in Saccharomyces cerevisiae. Genetics 163, 843-856.
Yu, X., and Gabriel, A. (2004). Reciprocal Translocations in Saccharomyces cerevisiae Formed by Nonhomologous End Joining. Genetics 166, 741-751.
Yvert, G., Brem, R. B., Whittle, J., Akey, J. M., Foss, E., Smith, E. N., Mackelprang, R., and Kruglyak, L. (2003). Trans-acting regulatory variation in Saccharomyces cerevisiae and the role of transcription factors. Nat Genet. 35, 57-64.

We claim:

1. A method for identifying the location in a source genome of a nonfixed genomic element, said nonfixed genomic element comprising a known nucleotide sequence, said method comprising:
(a) providing a population of genomic nucleic acid fragments; the population including a fragment that comprises the known nucleotide sequence or a portion thereof;
(b) contacting said population of genomic nucleic acid fragments with a targeting element such that the targeting element selectively binds at least a portion of the known nucleotide sequence to form a targeting element-genomic nucleic acid fragment complex; wherein said targeting element either has a separation group already attached before it is contacted with the genomic nucleic acid fragments or, if it does not, a separation group is attached after binding the targeting element to the known nucleotide sequence;
(c) immobilizing the targeting element-genomic nucleic acid fragment complex via the separation group to a substrate to form an immobilized complex,
(d) separating the immobilized complex from non-complexed genomic nucleic acid fragments,
(e) releasing the immobilized complex from the substrate;
(f) preparing a labeled probe by a method which uses the genomic nucleic acid fragments obtained in step (e) as template;
(g) applying the labeled probe to an array comprising immobilized nucleic acid molecules having nucleic acid sequences corresponding to known locations of a source genome under conditions which permit hybridization between the labeled probe and immobilized nucleic acid molecules having sufficient complementary sequence; and
(h) detecting hybridized labeled probes,
thereby identifying the location of the nonfixed genomic element.

2. The method of claim 1, wherein said nonfixed genomic element is a transposable element, a chromosomal rearrangement breakpoint, or a viral insertion.

3. The method of claim 1, wherein said targeting element comprises a nucleic acid sequence.

4. The method of claim 3, wherein said targeting element is an oligonucleotide that is complementary to a sequence contained in the known nucleotide sequence of the nonfixed genomic element.

5. The method of claim 1, wherein the labeled probes are fluorescently labeled.

6. The method of claim 1, wherein the separation group is selectively attached to the targeting element or an extension product of the targeting element in the presence of a polymerase after the targeting element specifically binds to all or a portion of the known nucleotide sequence to form a targeting element-genomic nucleic acid fragment complex.

7. The method of claim 6, wherein the targeting element is an oligonucleotide with an extendable 3′ hydroxyl terminus and the separation group is an immobilizable nucleotide and further wherein the separation group is attached to the targeting element by extending the oligonucleotide with a polymerase in the presence of the immobilizable nucleotide, thereby forming an extended oligonucleotide primer containing the immobilizable nucleotide.

8. The method of claim 7, wherein the immobilizable nucleotide is a biotinylated nucleotide.

9. The method of claim 1 wherein PCR amplification is not used to prepare labeled probes.

10. The method of claim 1, wherein the labeled probes are prepared by linear amplification and fluorescent labeling of the nucleic acid fragments obtained from the immobilized target element-genomic nucleic acid fragment complexes.

11. A method for identifying the location in a source genome of a repeated genomic element, said repeated genomic element comprising a known nucleotide sequence, said method comprising:
(a) providing a population of genomic nucleic acid fragments; the population including a fragment that comprises the known nucleotide sequence or a portion thereof;
(b) contacting said population of genomic nucleic acid fragments with a targeting element such that the targeting element selectively binds at least a portion of the known nucleotide sequence to form a targeting element-genomic nucleic acid fragment complex; wherein said targeting element either has a separation group already attached before it is contacted with the genomic nucleic acid fragments or, if it does not, a separation group is attached after binding the targeting element to the known nucleotide sequence;
(c) immobilizing the targeting element-genomic nucleic acid fragment complex via the separation group to a substrate to form an immobilized complex,
(d) separating the immobilized complex from non-complexed genomic nucleic acid fragments,
(e) releasing the immobilized complex from the substrate;
(f) preparing a labeled probe by a method which uses the genomic nucleic acid fragments obtained in step (e) as template;
(g) applying the labeled probe to an array comprising immobilized nucleic acid molecules having nucleic acid sequences corresponding to known locations of a source genome under conditions which permit hybridization between the labeled probe and immobilized nucleic acid molecules having sufficient complementary sequence; and
(h) detecting hybridized labeled probes,
thereby identifying the location of the repeated genomic element.

12. The method of claim 11, wherein said targeting element comprises a nucleic acid sequence.

13. The method of claim 12, wherein said targeting element is an oligonucleotide that is complementary to a sequence contained in the known nucleotide sequence of the repeated genomic element.

14. The method of claim 11 wherein PCR amplification is not used to prepare labeled probes.


SEQUENCE LISTING (9 sequences)

SEQ ID NO: 1 - DNA, 35 bp, Artificial sequence
  Probe YBLWTy1-1
gtagcgcctgtgcttcggttacttctaaagaagtc

SEQ ID NO: 2 - DNA, 35 bp, Artificial sequence
  Probe YBLWTy1-1
acaacacctccctcatctgctgttccagagaacca

SEQ ID NO: 3 - DNA, 35 bp, Artificial sequence
  Probe YBLWTy1-1
ctattatactacatcaacacacttgcacaacatat

SEQ ID NO: 4 - DNA, 35 bp, Artificial sequence
  Probe YBLWTy1-1RC
tttggaagctgaaatgtctaacggatcttgagttg

SEQ ID NO: 5 - DNA, 35 bp, Artificial sequence
  Probe YBLWTy1-1
caactcaagatccgttagacatttcagcttccaaa

SEQ ID NO: 6 - DNA, 35 bp, Artificial sequence
  Probe YBLWTy1-1RC
gaggcatgatgatggttctctggaacagcagatga

SEQ ID NO: 7 - DNA, 35 bp, Artificial sequence
  ProbeYBLWTy1-1
tcatctgctgttccagagaaccatcatcatgcctc

SEQ ID NO: 8 - DNA, 35 bp, Artificial sequence
  Probe YBLWTy1-1RC
ttggacggaaatagtatatgttgtgcaagtgtgtt

SEQ ID NO: 9 - DNA, 35 bp, Artificial sequence
  Probe YBLWTy1-1
aacacacttgcacaacatatactatttccgtccaa